(19) United States
(12) Patent Application Publication
(10) Pub. No.: US 2021/0316992 A1
(43) Pub. Date: Oct. 14, 2021

(54) Production of graphene structures

(71) Applicant: Teknologian tutkimuskeskus VTT Oy, Espoo (FI)

(72) Inventors: Katri Kurppa, Espoo (FI); Arja Paananen, Espoo (FI); Geza Szilvay, Espoo (FI); Timo Pulli, Espoo (FI); Henrik Sandberg, Espoo (FI); Miika Soikkeli, Espoo (FI); Jukka Ketoja, Espoo (FI); Teemu Ruotsalainen, Espoo (FI)

(21) Appl. No.: 17/051,808

(22) PCT Filed: Apr. 26, 2019
(86) PCT No.: PCT/FI2019/050339

§ 371 Date: Oct. 30, 2020

(30) Foreign Application Priority Data
Apr. 30, 2018 (FI) 20185400

(51) Int. Cl. C01B 32/192; C01B 32/198

(52) U.S. Cl. CPC C01B 32/192; B82Y 40/00; C01P 2004/03; C01B 32/198

(57) ABSTRACT

According to an example aspect of the present invention, there is provided a method for producing graphene foam structures by assembling graphene or graphene oxide in a three-dimensional structure, wherein biomolecular surface active agents are used as a template in a water-based foam. The graphene foam structures produced by the method of the invention find industrial application for example in sensing and material applications.

### FIELD

The present invention relates to a method of producing three-dimensional graphene foam structures by assembling graphene, particularly graphene oxide, into a water-based foam using a biomolecular surface active agent, and then optionally reducing the structure at high temperatures to graphene. The invention also relates to graphene structures produced by the method of the invention.

### BACKGROUND

Graphene is a crystalline allotrope form of carbon consisting of a single layer of carbon atoms arranged in a hexagonal lattice. Graphene has unique electrical properties, such as high charge carrier mobility, which are promising for electronic applications. Graphene has also excellent mechanical properties and has therefore been suggested for several applications on the basis of its strength, for example.

Graphene foams (GF) are materials with three-dimensional (3D) cellular structure consisting of two-dimensional (2D) graphene nanosheets. Due to their cellular structure, graphene foams possess unique properties, such as high porosity, low density, and excellent elasticity. The properties of graphene, including a large specific surface area, good electrical conductivity, and high chemical and thermal stability, are also among the properties of graphene foams. Consequently, graphene foams have found applications for example in energy conversion and storage, catalysis, sensing, and pollution control.

Graphene oxide (GO), in particular graphene oxide flakes, can be used to form a three-dimensional graphene foam. The graphene foam is ultralight, viscoelastic and conductive and is a potential material for use in diverse sensing and material applications, for example in pressure sensing or 3D electrode setups in fuel cells. For the material to be usable, properties such as cell size and morphology need to be controlled. In addition, processability of the graphene foam needs to be improved for more flexible use in e.g. printing technologies or in situ applications. To improve the possibilities of modifying and handling graphene oxide, the stability (material lifetime) of the template needs to be enhanced.

Current technologies for manufacturing graphene foams include chemical vapour deposition (CVD) and hydrothermal reaction. In chemical vapour deposition, a metal foam or a three-dimensional mesh of metal filaments is coated by graphene, and the metal, for example nickel, is removed. Graphene foams can also be prepared via the 3D self-assembly of graphene oxide (GO) or chemically converted graphene (CCG) during hydrothermal reaction, chemical reduction, or direct lyophilisation-thermal reduction.

The method disclosed by Bai et al (2015) involves mechanically foaming a graphene oxide dispersion with the assistance of a surfactant, followed by lyophilisation and thermal reduction. The graphene oxide dispersion is first mixed with an anionic surfactant, sodium dodecyl sulphate (SDS), and the dispersion is foamed to a viscous foam. The foamed dispersion is then frozen in liquid nitrogen and lyophilized to yield a GO foam (GOF). During pyrolysis at a temperature of 400° C. under an argon atmosphere for 2 h, the GOF was converted into a GF.

CN 105384165A discloses a preparation method of graphene aerogel, wherein a surfactant is added to a graphene oxide dispersion to obtain a graphene oxide foam suspension, followed by freezing and freeze drying the suspension to obtain the graphene aerogel material. WO 2018/001206 A1 relates to a method for producing graphene-based capacitive carbon, wherein a nitrogen-containing compound, such as urea or ethylenediamine, is used as a foaming agent.

Barg et al (2014) studied assembly of chemically modified graphene into complex cellular networks. They prepared highly concentrated GO suspensions in water at neutral pH but just before emulsification, they adjusted the suspension pH between 2 and 3. After emulsification, the GO emulsion is frozen and subsequently the solvents are eliminated by freeze drying. The final step is a thermal treatment in a reducing atmosphere to promote the reduction of GO.

The above attempts wherein detergent foams or emulsions of aqueous solution and organic solvents are used as a template for graphene oxide assembly have been created in order to achieve simple, scalable and processable route to produce graphene foams. However, the structure of the SDS template is unstable and requires rapid lyophilisation to fix the composite structure. Therefore, processability has been improved by using a water/oil (decane or toluene) emulsion system. However, fixation of the structures still requires specialized freezing techniques. In addition, solvent based systems do not promote to a sustainable solution. Solvent containing solutions are neither safe for household use (i.e. self-applicable, injectable graphene).

Hydrophobins have raised interest for example as special biosurfactants and as foaming agents. Hydrophobins are a group of small, amphiphilic surface active proteins that are expressed by filamentous fungi. They have a remarkable surface activity and thus their influence on the stability of bubble dispersions of the food industry has been of interest. Two classes of hydrophobins have been distinguished based on hydrophobicity profiles and aqueous solubilities: Class I hydrophobins, forming highly insoluble aggregates which can only be dissolved with strong acids; and Class II hydrophobins, which can be readily dissolved in aqueous solutions.

The foam stability of aerated solutions containing the Class II hydrophobin protein HFBII from Trichoderma reesei has been investigated and compared with that of other typical food emulsifiers and aerating agents by Cox et al (2009). Hydrophobin foams were found to be stable for several months or even for years, which is well in excess of the stability of foams stabilised using other food-aerating/emulsifying agents.

Gravagnuolo et al (2015) have studied ultrasonication-based production of biofunctionalized microsheets of graphene using a Class I hydrophobin, namely Vmh2 from the edible white-rot fungus Pleurotus ostreatus. They used liquid phase ultrasonic exfoliation of raw graphitic material assisted by hydrophobin Vmh2 and produced biofunctionalized few-layer graphene flakes.

However, examples of using hydrophobin stabilised foams for nanoparticle assembly have not been described in the prior art, nor is there a disclosure of graphene foam production using any biomolecular template. Moreover, the graphene foams described in the prior art are not sufficiently stable and thus lack processability needed for most applications.

### SUMMARY OF THE INVENTION

The invention is defined by the features of the independent claims. Some specific embodiments are defined in the dependent claims.

According to a first aspect of the present invention, it is provided herein a novel method for producing graphene foam structures by assembling graphene, particularly graphene oxide, into a three-dimensional structure, wherein water-based foam comprising a biomolecular surface active agent is used as a template.

According to a second aspect of the present invention, it is provided herein a graphene foam structure, preferably an electrically conductive graphene foam structure, which has been produced by the method of the invention.

According to a further aspect, the invention provides the use of the graphene foam structures produced by the method of the invention in pressure sensing applications, in biosensing, in printing technologies, in energy conversion and storage, or as electrode material.

According to an even further aspect, there is provided a method for improving stability of graphene foams, wherein surface active proteins, such as hydrophobins, are used as a template for assembling graphene or graphene oxide nanoparticles.

The present invention thus aims at producing stable three-dimensional graphene structures or foams. In contrast to technologies described in prior art, this invention uses a novel method wherein surface active protein stabilized foams are used as a template for assembling graphene, particularly graphene oxide, more particularly graphene oxide flakes or nanoparticles.

In one aspect, the present invention thus provides graphene foams and graphene structures with high-performance properties, produced with cost-effective process choices.

Considerable advantages are achieved by means of the present invention. The method of the invention improves processability and modification possibilities of graphene foams. Due to the extreme stability of the foams obtained by means of the present invention, graphene composite foams may be handled and applied in a more flexible manner, according to the application needed. The longer lifetime of graphene foam enhances especially the integration of graphene foam in applications and broadens its usability by different techniques.

The present invention thus provides a completely new method of creating self-standing, three dimensional graphene structures. The composition of the graphene foams can be adjusted due to the extreme stability of the template. Important parameters include cell size of the foam, morphology of the material and graphene content, all contributing to the overall conductivity and viscoelasticity of the graphene foam.

Next, the present technology will be described more closely with reference to the drawings and certain embodiments.

### BRIEF DESCRIPTION OF THE DRAWINGS

FIG. 1 illustrates results of graphene oxide foam production using a hydrophobin HFBI stabilized foam template. An SDS templated graphene foam is used as a reference. SDS foam has completely disintegrated after 2.5 h while HFBI template foams are intact on the next day.

FIG. 2 illustrates SEM images of HFBI-GO foam (sample C, FIG. 1).

FIG. 3 comprises a Table summarizing the results obtained by different samples, foaming techniques, graphene oxide/hydrophobin ratios, and drying methods. “Sup” refers to heat treated supernatant containing hydrophobin, “Pre-foam” means that graphene oxide was added to pre-foamed protein solution. The reference sample “SDS+ GO” was prepared as Bai et al. “FD” refers to freeze-drying. The smaller resistance in volume resistance measurements refers to better conductivity. Specific surface areas were measured by BET method (Brunauer et al, 1938).

FIG. 4 illustrates foam samples prepared by different methods, sample letters are according to samples listed in table (FIG. 3). a) Sample F is made of supernatant containing hydrophobin HFBI-4550 and GO, where the ratio in foam is 2 mg/ml of GO/0.15 mg/ml HFBI-4550. b) Sample G is SDS-GO foam prepared as Bai et al, where the corresponding ratio is 4/1 (SDS). c) and d) In sample I the protein concentration was increased by adding purified HFBI-4550 to the supernatant, and the GO/protein ratio was 3/1. By increasing the protein amount an ultralight conductive foam was obtained.

FIG. 5 illustrates SEM images of foam samples prepared by different methods, sample letters are according to samples listed in table (FIG. 3). a) and b) Sample J is made of supernatant containing hydrophobin HFBI-4550 and GO, where the ratio in foam is 3 mg/ml of GO/0.075 mg/ml HFBI-4550. c) and d) Sample L is made of pure HFBI and GO, where the corresponding GO/protein ratio is 3/1. e) and f) Sample M is made by adding purified HFBI-4550 to the supernatant before mixing with GO. The GO/protein ratio is 3/1.2.

FIG. 6 illustrates volume resistance values of (a) a commercial graphene aerogel and (b) the graphene foam according to the present invention (sample I, FIG. 3) when conductivity was measured during compression. The volume resistance value decreases, i.e. conductivity increases, during compression. At highest compression the volume resistance increases, which may originate from partial breakdown of the graphene foam structure.

FIG. 7 illustrates measurements of compression in relation to applied force. Measurements were carried out by applying standard weights on top of GFoam pilars and the compression of the sample was measured. Sample L (dashed lines) is made of pure HFBI and GO, where the corresponding GO/protein ratio is 3/1. Sample M (long dashed lines) is made by adding purified HFBI-4550 to the supernatant before mixing with GO. The GO/protein ratio is 3/1.2. Sample I (solid lines) is made by adding purified HFBI-4550 to the supernatant before mixing with GO. The GO/protein ratio is 3/1. Sample letters are according to samples listed in table (FIG. 3). Measurements were repeated twice (black and gray lines for each sample). Based on the Compression vs Force graph it could be estimated that the Young's modulus for GFoam material (Sample M) is approximately 25 kPa (corresponding value for fibre foam is 200 mPa and for paper 5 GPa). The material is very soft, mechanical properties are comparable to cotton wool, compression recovery is about 80%.

### EMBODIMENTS

In the present context, “graphene” refers generally to material that consists essentially of a one-atom-thick planar sheet of sp² bonded carbon atoms. In graphene, the carbon atoms are densely packed in a honeycomb crystal lattice.

“Graphene foam” and “graphene foam structure” refer generally to three-dimensional cellular graphene structures consisting of two-dimensional graphene nanosheets. In embodiments of the invention, “graphene foam” or “graphene foam structure” may refer also to wet graphene oxide foams obtained by method of the invention.

“Graphene oxide” refers to oxidized form of graphene, a single-atomic layered material laced with oxygen-containing groups. Graphene oxide is easily dispersed in water and other organic solvents, and has a low electrical conductivity.

“Biomolecular surface active agent” refers generally to surface active proteins, for instance natural proteins from fungi or any modified or synthetically produced polypeptide that is functionally equivalent to surface active proteins in achieving the desired effect. A biomolecular surface active agent thus includes but is not limited to surface active proteins, preferably amphiphilic proteins, particularly hydrophobins.

In the present context, “template” or “biotemplate” refers to a three-dimensional structure which comprises water-based foam of a biomolecular surface active agent or a surface active protein. Said template forms the basis of the graphene foam structures to be prepared by the method of the invention. The biomolecular surface active agent can be removed from the final graphene foam structure for example by pyrolysis or by laser techniques.

The present invention is based on the finding that graphene foams can be produced by assembling graphene, particularly graphene oxide, for example in the form of graphene oxide flakes, in a water-based foam using a surface active protein, such as a hydrophobin. Surface active proteins, particularly hydrophobins, assemble at the interface of air and water to form highly viscoelastic, monomolecular films. Due to the chemical functionalities of graphene oxide, the graphene oxide flakes are able to interact with chemical groups at the protein surface. This allows the graphene oxide flakes to be positioned in the continuous phase of the foam.

In one embodiment of the present invention, the method for producing graphene foam structures by assembling graphene oxide in a 3D structure by using at least one biomolecular surface active agent comprises:
preparing a water-based foam of the biomolecular surface active agent; and
mixing graphene oxide in the water-based foam of the biomolecular surface active agent, and preferably foaming simultaneously to create a dense foam.

Optionally, in the above embodiment also an additional foaming of the mixture comprising the GO and the biomolecular surface active agent is possible to obtain a larger foam volume.

Foaming can be carried out by any suitable method, for example by mechanical mixing or by bubbling the solution with nitrogen gas. When “a dense foam” comprising graphene oxide or graphene and the biomolecular surface active agent is prepared, the dense foam generally refers to a foam with an air content below 65%. Typically in “a foam with a high air content”, the air content can be as high as about 85%.

In another embodiment of the invention, the method for producing graphene foam structures by assembling graphene oxide in a 3D structure by using at least one biomolecular surface active agent comprises at least the following steps:
mixing a water solution of the biomolecular surface active agent and a graphene oxide water dispersion to obtain a solution comprising the biomolecular surface active agent and the graphene oxide;
foaming the solution comprising the biomolecular surface active agent and the graphene oxide to a dense foam, preferably by bubbling or by mechanically mixing said solution until no additional foam is formed.

In the above embodiments, the graphene oxide is preferably in the form of graphene oxide dispersion, which preferably comprises graphene oxide in a concentration of 1 to 7 mg/ml, or 2 to 5 mg/ml, for example 2, 3 or 4 mg/ml.

When the water solution of the biomolecular surface active agent and the graphene oxide water dispersion are mixed before foaming, they are preferably mixed in a ratio of 1:10 to 1:1, for example in a ratio of approximately 1:3.

In an embodiment of the invention, the concentration of the biomolecular surface active agent in the water solution of the biomolecular active agent is 0.15 to 5 mg/ml, 0.5 to 5 mg/ml or 1 to 5 mg/ml, for example 2 to 4 mg/ml.

In a further embodiment of the invention, the method for producing graphene foam structures by assembling graphene oxide in a 3D structure by using a biomolecular surface active agent as a template in a water-based foam comprises the steps of:
dispersing graphene oxide in a solution of the biomolecular surface active agent,
optionally exposing the dispersion to ultrasonic waves to facilitate exfoliation,
foaming the dispersion comprising the graphene oxide and the biomolecular surface active agent to a dense foam, preferably by bubbling or mechanically mixing said dispersion until no additional foam is formed.

In all the above embodiments the method of the invention preferably further comprises the steps of drying the obtained graphene oxide foam, and exposing the dried graphene oxide foam to pyrolysis to at least partially reduce graphene oxide to graphene. For applications where conductivity is desired, the reduction step is required for the graphene foam structure to be conductive.

Graphene to be assembled in the three-dimensional structure according to the method of the invention is preferably at least partly exfoliated. For the purposes of the invention graphene is preferably in the form of graphene oxide flakes, graphene oxide nanoparticles, graphene oxide water dispersion, graphene oxide nanopowder, reduced graphene oxide powder, or single layer graphene oxide.

In order to ensure a high degree of exfoliated graphene oxide, in an embodiment of the invention the graphene oxide flakes or the graphene oxide water dispersion are preferably subjected to ultrasonication and homogenization.

In a further embodiment of the invention, graphene is exfoliated by hydrophobins as described in detail in WO 2010/097517 A1. The graphene exfoliated by hydrophobins can be used instead of or in addition to graphene oxide for preparing graphene foam structures according to any one of the above described embodiments.

### Biomolecular Surface Active Agents

The biomolecular surface active agents may be surface active proteins, for instance natural proteins from fungi or any modified or synthetically produced polypeptide that is functionally equivalent to surface active proteins in achieving the desired effect. The proteins may also be fusion proteins.

According to an embodiment, the proteins include proteins that contain a part that is more hydrophobic than the rest of the protein's body. In another embodiment, the proteins are proteins that have a hydrophobic part that is capable of adhering to the surface of graphene. According to a further embodiment, proteins include amphiphilic proteins. Particularly preferred examples of such amphiphilic proteins include hydrophobins. Also other proteins, such as rodlins, chaplins, and ranaspumin, can exhibit such properties.

In preferred embodiments, the proteins include hydrophobins, particularly class II hydrophobins. Examples of class II hydrophobins include HFBI, HFBII, HFBIII, and other polypeptides that have resemblance in properties or sequence to said polypeptides. Examples of hydrophobins include therefore also other similar polypeptides which have corresponding properties.

In nature, hydrophobins have been found as amphiphilic proteins produced by filamentous fungi. However, recombinant DNA technologies allow their production in a variety of other organisms such as bacteria, archea, yeasts, plant cells, or other higher eucaryotes. Hydrohophobins may also be produced without the use of living cells, either by synthesis or by cell-free production methods. In addition to adhesive property, these hydrophobins have also some further useful properties that can be utilized in some embodiments. For example, hydrophobins of this type are typically able to form protein films, which can be used to support the exfoliated graphene, for instance.

The proteins in embodiments can also include fusion proteins that comprise at least two functional parts. One of the functional parts can be selected such that it has ability to adhere to graphene whereas at least one of the other parts can be selected according to other desired functions. Such other desired functions may relate, for example, to solubility, electrical properties, mechanical properties, chemical properties and/or adhesive properties.

According to the needs of the application, class I and/or class II hydrophobins can be used. The class I hydrophobins typically form aggregates that are highly insoluble, whereas the aggregates of class II members dissolve more readily. This information can be used when selecting suitable proteins according to the needs of each application. As stated above, class II hydrophobins are preferred in the method of the invention.

Examples of class II hydrophobins include HFBI, HFBII, and HFBIII that can be obtained from Trichoderma reesei. Other sources of hydrophobins than Trichoderma include all filamentous fungi, such as Schizophyllum, Aspergillus, Fusarium, Cladosporium, and Agaricus species. In the present invention, class II hydrophobins produced by Trichoderma reesei are preferred, such as HFBI or HFBII produced by Trichoderma reesei, particularly hydrophobin HFBI, such as HFBI-4550 (WO 2015/082772 A1).

The hydrophobins for use in the present invention can be produced by fermentation as described for example by Linder et al (2001). The hydrophobins obtained after fermentation of for example Trichoderma reesei can be purified at different levels and used in the method of invention. Thus by using a suitable preparation process, hydrophobin solutions of lower purity grade may also be used for formation of the graphene foams. This lowers costs and simplifies down-stream processing. In an embodiment, other proteins than hydrophobins (background proteins) can be removed for example by heating.

In an embodiment of the invention, a hydrophobin-containing supernatant from the fermentation of for example Trichoderma reesei is used as such or preferably after heat treatment for formation of a graphene foam.

In a further embodiment, purified hydrophobin is added to a hydrophobin-containing supernatant or to a heat-treated hydrophobin-containing supernatant before the supernatant is used for formation of a graphene foam. Addition of purified hydrophobin increases the hydrophobin content of the supernatant. Alternatively, the heat-treated supernatant can be concentrated to increase the hydrophobin content.

According to an embodiment of the invention, the graphene foam can be obtained at the wet-stage of the present method. The graphene foam obtained at the wet-stage is easily moldable, stable, maintains its volume at least for several days and can be cut with a scalpel. In an embodiment, the wet graphene foam obtained by method of the invention is used as such in printing applications or it may be molded and dried in shape.

In a further embodiment, the graphene foam obtained at the wet-stage of the present method is dried, for example at a room temperature or at a higher temperature, preferably at a temperature of 30 to 100° C., preferably at least at about 60° C., for example at 60-70° C., or by freezing and freeze-drying.

In a preferred embodiment of the invention, the dried graphene foam is exposed to pyrolysis. During pyrolysis at least part of the graphene oxide is reduced to graphene. Further, during pyrolysis the biotemplate (biomolecular surface active agent) is removed.

The step of pyrolysis is preferably carried out at 350 to 900° C., or at about 400 to 800° C., for example at a temperature of at least 400° C. For applications requiring conductivity of the graphene foam structure, the step of pyrolysis is preferred. In an embodiment of the invention, the temperature is raised to the desired pyrolysis temperature at a rate of 10° C./min. The pyrolysis temperature is maintained for example for 1 to 4 hours, such as for about 2 hours, preferably under nitrogen flow.

In a further embodiment of the invention, the biotemplate can be removed by laser techniques.

As discussed above, in embodiments the present invention thus provides a method for producing graphene foam structures by assembling graphene oxide in a water-based foam using a biomolecular surface active agent, preferably a hydrophobin, wherein the method comprises
preparing a water-based foam of the biomolecular surface active agent, and mixing graphene oxide in the water-based foam of the biomolecular surface active agent, while preferably foaming simultaneously to create a dense foam; or
mixing a water solution of the biomolecular surface active agent and a graphene oxide water dispersion to obtain a solution comprising the biomolecular surface active agent and the graphene oxide, and foaming the solution comprising the biomolecular surface active agent and the graphene oxide to create a dense foam, preferably by bubbling or mechanically mixing said solution until no additional foam is formed; or
dispersing graphene in the form of graphene oxide in a solution of the biomolecular surface active agent, optionally exposing the dispersion to ultrasonic waves to facilitate exfoliation, and foaming the dispersion comprising the graphene oxide and the biomolecular surface active agent to a dense foam, preferably by bubbling or mechanically mixing said dispersion until no additional foam is formed.

The wet graphene foam structure prepared according to any of the embodiments described above can be used as such for example in printing applications or it can be molded and dried in shape. In a preferred embodiment the wet graphene foam structure is dried and exposed to pyrolysis as discussed above.

In an embodiment, the invention thus provides graphene foam structures produced by the method of the invention, either in a wet form or in a dried and pyrolysed form. The invention also provides the use of said graphene foam structures in pressure sensing applications, in biosensing, in printing technologies, in energy conversion and storage, in catalysis, in pollution control or as electrode material.

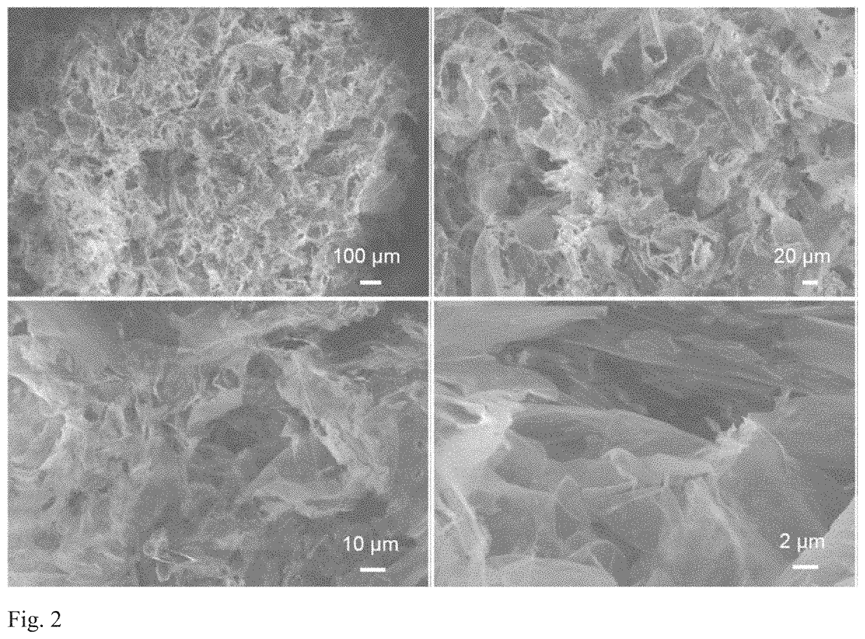

The dried and pyrolysed graphene foam structure prepared by the method of the invention is highly porous, sensitive, low density material, which has a good electrical conductivity as well as high chemical and thermal stability. In an embodiment, the pyrolysed graphene foam structures prepared by the method of the invention have a density of about 1 to 10 g/dm³, particularly about 2.5 g/dm³. This means that the graphene foam structures according to the invention are approximately 30 times lighter than Styrofoam. They also have a large specific graphene surface area, such as for example 35-40 m²/g, particularly about 38 m²/g. The graphene foam structures prepared by the method of the invention have a cell size of the foam and cell morphology as exemplified in the SEM images (FIG. 2).

Further, the electrical conductivity values of the graphene foam structures of the invention show a volume resistance of approximately 500 to 5000 Ohmcm, or about 500 to 3500 Ohmcm. Said volume resistances are quite reasonable for the intended applications, wherein a very high conductivity means less sensitivity, such as in pressure sensing applications for instance. For comparison, a commercial graphene aerogel has a volume resistance of 900 Ohmcm.

Another advantageous feature of the graphene foam structures according to the invention is that they are touch sensitive, resilient and responsive materials. As an example of resiliency, the graphene foam structures of the invention show 80-100% recovery after compression. During compression resistance of the material decreases (conductivity increases), making the graphene foam structures of the invention particularly suitable for pressure sensing applications.

The dried and pyrolysed graphene foam structure prepared by the method of the invention possesses pressure sensitive conductivity which makes it particularly suitable for pressure sensing applications.

### EXPERIMENTAL

A 0.1% solution of HFBI was used to solubilize graphene oxide flakes in buffer solution by mild sonication in a water bath. The resulting solution was subjected to ultrasonication and homogenization to ensure a high degree of exfoliated graphene oxide flakes. To trigger massive foam formation, the solution was bubbled with nitrogen gas until no additional foam was formed.

In other embodiment, dry graphene oxide powder was mixed in pre-prepared HFBI foam by mixing with nitrogen gas.

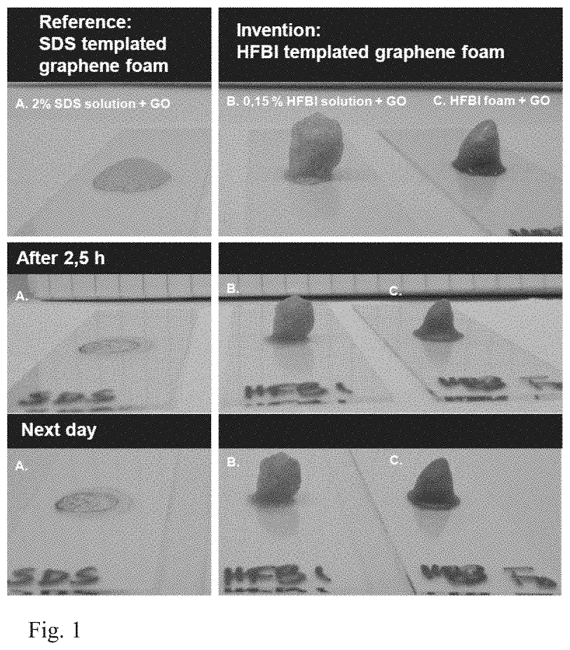

The foaming solution was finally shaken to create a highly dense, grey foam. The foam was observed to be stable (without reduction in volume) for at least 3 days in a closed test tube. The foam could be handled and foam chunks could be applied on a glass support using a spoon. The deposited foam samples held their shape for at least one day, with only minor shrinkage observed. The foam produced from a pre-prepared hydrophobin foam could be gently cut with a scalpel and handled without excessive crumbling (see FIG. 1).

As a reference, a 2% SDS solution was used to create the foam template with graphene oxide. In this case the produced foam was white and highly unstable. The foam started to degrade immediately and could not be handled. Deposition on a glass side resulted in a weak foam drop, which dried completely during 2 hours (see FIG. 1). The foam could only be applied by a pipette due to its wetness. The structure of the dry foam is porous (see FIG. 2).

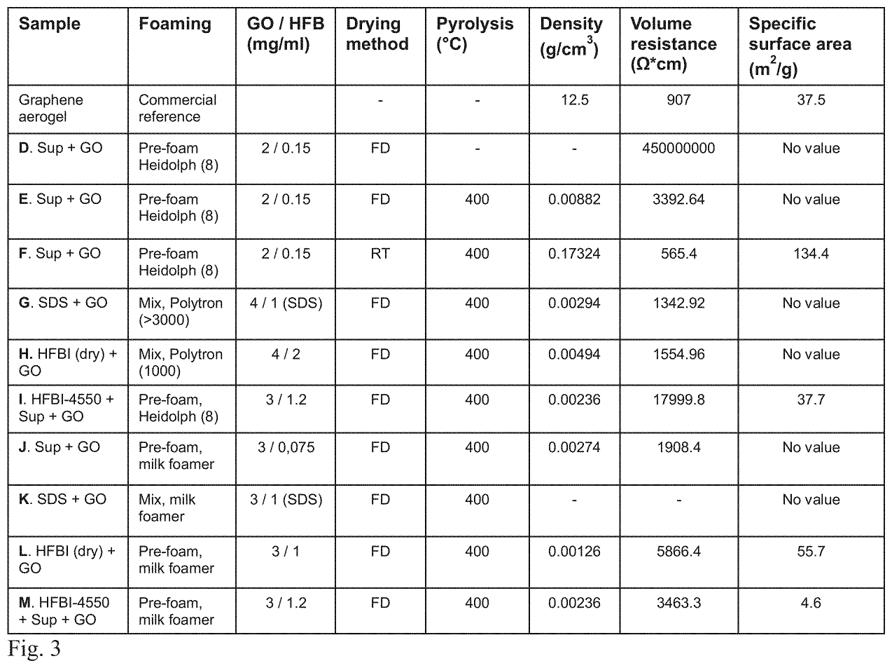

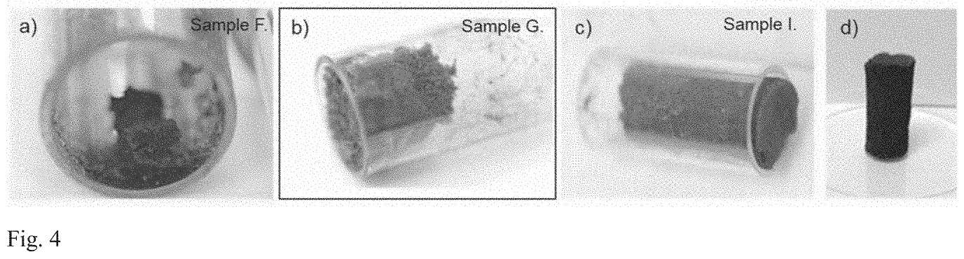

Foam preparation was further developed to make more dense foam that would keep the shape and structure during pyrolysis. FIG. 3 shows a table of foams made by different methods and of different compositions. GO content in the mixture before drying is preferably at least 3 mg/ml, and protein concentration preferably at least 1 mg/ml. Typically 5-10 ml of protein sample was foamed first to make a pre-foam and GO was then gradually added during further foaming. Freeze-drying was preferably required as a drying step to avoid shrinkage of the structure. Pyrolysis at least at 400° C. was required to obtain conductive material. FIG. 4 shows images of graphene foams after pyrolysis.

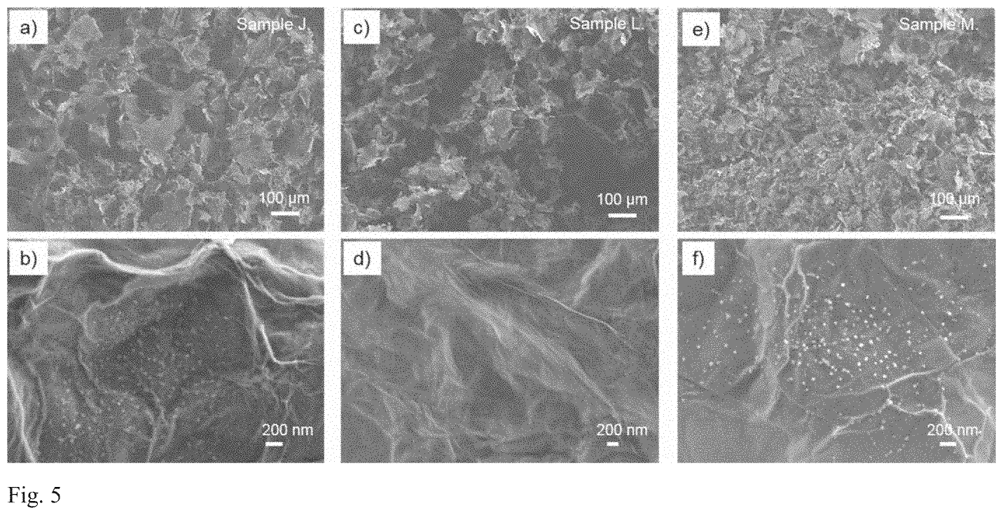

FIG. 5 shows SEM images of pyrolyzed graphene foams prepared by the above described method. The structures are porous. In order to reduce the preparation costs, as low purity level protein samples as possible were used. The protein samples were heat-treated supernatants, where heat treatment was done to precipitate the majority of other proteins in the supernatant. The heat-treated supernatant contained approximately 300 mg/L hydrophobin, and hence purified protein was added to increase the hydrophobin content. Another option is to concentrate the heat-treated supernatant. Supernatant samples contain also salts and sugars, of which salts can remain after pyrolysis.

The porosity and the specific surface areas were determined for the graphene foams. The specific surface areas were determined by Micromeritics TriStar 3000 gas adsorption analyzer using Brunauer-Emmett-Teller (BET) surface area analysis theory (Brunauer et al, 1938). In comparison to commercial graphene aerogel, similar values were obtained.

Results from Conductivity Measurements

In the conductivity measurements, samples were placed between gold electrodes and the compression was adjusted using micrometer screw. The resistance of the material was measured with 2 mm steps either using Keithley 6517A electrometer high resistance meter or Vellemann DVM891 multifunctional digital multimeter.

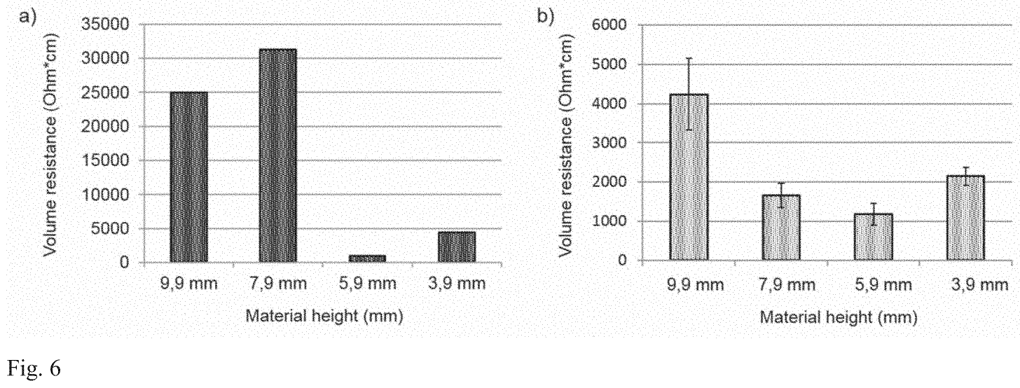

Conductivity measurements are illustrated in FIG. 6. Resistance decreases, i.e. conductivity increases, when material is compressed. When comparing the graphene foam according to the invention to a commercial graphene aerogel, it was found that the materials behaved similarly during compression but the graphene foam according to the invention was more conductive.

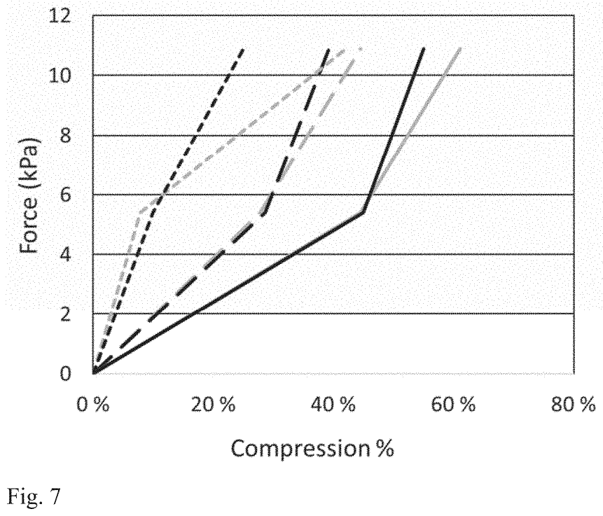

Measurements of compression in relation to applied force are illustrated in FIG. 7. Measurements were carried out by applying standard weights on top graphene foam pilars and the compression of the sample was measured. Based on the Compression vs Force graph it could be estimated that the Young's modulus for GFoam material (Sample M) is approximately 25 kPa (corresponding value for fibre foam is 200 mPa and for paper 5 GPa). The material is very soft, mechanical properties are comparable to cotton wool, compression recovery is about 80%.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

The present method and the products thereby produced find industrial application for example in sensing and material applications, such as pressure sensing or 3D electrode setups in fuel cells, energy storage, or in printing technologies. The formed foam composite may be used as such or molded and dried in shape. At least some embodiments of the present invention find industrial application also in energy conversion and storage, in catalysis, in biosensing, in pollution control or as electrode material.

### ACRONYMS LIST

BET Brunauer-Emmett-Teller method
CCG Chemically converted graphene
CVD Chemical vapor deposition
FD Freeze drying, freeze-dried
GF Graphene foam
GO Graphene oxide
GOF Graphene oxide foam

### HFB Hydrophobin

RT Room temperature
SDS Sodium dodecyl sulphate
SEM Scanning electron microscope

### CITATION LIST

### Patent Literature

CN105384165 A
WO 2015/082772 A1
WO 2018/001206 A1

### Non Patent Literature

Bai J. et al (2015). Ultra-light and elastic graphene foams with a hierarchical structure and a high oil absorption capacity. J. Mater. Chem. A, 3, 22687.
Barg, S. et al (2014). Mesocale assembly of chemically modified graphene into complex cellular networks. Nature Communications, 5:4328.
Brunauer, S. et al (1938). “Adsorption of Gases in Multimolecular Layers”. Journal of the American Chemical Society. 60 (2): 309-319.
Cox, A. et al. (2009). Exceptional stability of food foams using class II hydrophobin HFBII. Food Hydrocolloids 23, 366-376.
Gravagnuolo, A. M. et al. (2015). In Situ Production of Biofunctionalized Few-Layer Defect-Free Microsheets of Graphene. Adv. Funct. Mater. 25, 2771-2779.
Linder, M. et al. (2001). The hydrophobins HFBI and HFBII from Trichoderma reesei showing efficient interactions with nonionic surfactants in aqueous two-phase systems. Biomacromolecules. Vol. 2, No: 2, 511-517.

1. A method for producing graphene foam structures by assembling graphene into a three-dimensional structure, wherein a water-based foam comprising a biomolecular surface active agent is used as a template for assembling the graphene.

2. The method according to claim 1, wherein the biomolecular surface active agent comprises an amphiphilic protein.

3. The method according to claim 1, wherein the biomolecular surface active agent comprises at least one hydrophobin.

4. The method according to claim 3, wherein the at least one hydrophobin is from Trichoderma reesei.

5. The method according to claim 1, wherein the biomolecular surface active agent comprises a hydrophobin-containing supernatant from the fermentation of filamentous fungi, wherein said supernatant is used as such or purified at different levels for formation of the water-based foam.

6. The method according to claim 5, wherein purified hydrophobin is added to the hydrophobin-containing supernatant before the supernatant is used for formation of the water-based foam.

7. The method according to claim 1, wherein the graphene is in the form of graphene oxide flakes, graphene oxide nanoparticles, graphene oxide water dispersion, graphene oxide nanopowder, graphene oxide powder, or single layer graphene oxide.

8. The method according to claim 1, wherein the method comprises:
preparing a water-based foam of the biomolecular surface active agent; and
mixing graphene oxide in the water-based foam of the biomolecular surface active agent.

9. The method according to claim 1, wherein the method comprises:
mixing a water solution of the biomolecular surface active agent and a graphene oxide water dispersion to obtain a solution comprising the biomolecular surface active agent and the graphene oxide, and
foaming the solution comprising the biomolecular surface active agent and the graphene oxide to a dense foam.

10. The method according to claim 8, wherein the graphene oxide is in the form of water dispersion, which preferably comprises graphene oxide in a concentration of 1 to 7 mg/ml.

11. The method according to claim 9, wherein the water solution of the biomolecular surface active agent and the graphene oxide water dispersion are mixed in a ratio of 1:10 to 1:1.

12. The method according to claim 1, wherein the method comprises:
dispersing graphene in the form of graphene oxide in a solution of the biomolecular surface active agent,
optionally exposing the dispersion to ultrasonic waves to facilitate exfoliation, and
foaming the dispersion comprising the graphene oxide and the biomolecular surface active agent to a dense foam.

13. The method according to claim 12, wherein the graphene oxide is in the form of graphene oxide flakes, graphene oxide nanoparticles, graphene oxide powder, or single layer graphene oxide.

14. The method according to claim 1, wherein the concentration of the biomolecular surface active agent in the water-based foam is 0.15 to 5 mg/ml.

15. The method according to claim 12, further comprising:
drying the obtained graphene oxide foam;
exposing the dried graphene oxide foam to pyrolysis to at least partially reduce graphene oxide to graphene.

16. The method according to claim 15, wherein the drying step comprises drying at room temperature or at a temperature of 30 to 100° C.

17. The method according to claim 15, wherein the pyrolysis step comprises pyrolysis at 350 to 900° C.

18. The method according to claim 1, wherein graphene, which has been exfoliated by hydrophobins, is used instead of or in addition to graphene or graphene oxide for preparing the graphene foam structures.

19. A graphene foam structure produced by the method according to claim 1.

20. (canceled)

21. (canceled)

22. (canceled)
